(19) United States
(12) Patent Application Publication
(10) Pub. No.: US 2025/0236827 A1
(43) Pub. Date: Jul. 24, 2025

(54) CELL CULTURE ASSISTING APPARATUS

(71) Applicant: Beyond Mind Co., Ltd., Taipei City (TW)

(72) Inventors: Chan-Guan KOH, Taipei City (TW); Zhuang-Sheng KOH, Taipei City (TW)

(21) Appl. No.: 18/965,787

(22) Filed: Dec. 2, 2024

Related U.S. Application Data
(60) Provisional application No. 63/623,463, filed on Jan. 22, 2024.

(30) Foreign Application Priority Data
Sep. 27, 2024 (TW) 113137072

(51) Int. Cl. C12M 1/00; C12M 3/00

(52) U.S. Cl. CPC C12M 23/02; C12M 23/44; C12M 29/20

(57) ABSTRACT

A cell culture assisting apparatus includes a stepped structure that has a first section, a second section, a third section, a first step, and a second step. A vent hole extends through the stepped structure and opening at bottom and top ends of the stepped structure. The first section surrounds the vent hole. The second section surround the vent hole below the first section and is wider than the first section. The third section surrounds the vent hole below the second section and is wider than the second section. The first step is formed around the vent hole between the first section and the second section. The second step is formed around the vent hole between the second section and the third section.

### CROSS-REFERENCE TO RELATED APPLICATION

This application claims priority to U.S. Provisional Patent Application No. 63/623,463, filed on Jan. 22, 2024, and Taiwanese Invention patent application No. 113137072, filed on Sep. 27, 2024, both of which are incorporated by reference herein in their entirety.

### FIELD

The disclosure relates to a cell culture assisting apparatus, and more particularly to a cell culture assisting apparatus capable of assisting cell culture.

### BACKGROUND

Conventionally, a cell growth medium is added to a petri dish containing cells in order to increase cell growth rates. However, the industry is always researching new equipment and techniques to increase cell growth rates, and technical advancement is always continuing in this area.

### SUMMARY

Therefore, an object of the disclosure is to provide a cell culture assisting apparatus that can alleviate at least one of the drawbacks of the prior art.

According to the disclosure, the cell culture assisting apparatus capable of assisting cell culture includes a stepped structure and a vent hole. The stepped structure has a top end, a bottom end, a first section, a second section, a third section, a first step, and a second step. The vent hole extends through the stepped structure and opening at the bottom and top ends. The first section surrounds the vent hole and defining the top end. The second section surround the vent hole below the first section and is wider than the first section. The third section surrounds the vent hole below the second section and is wider than the second section. The first step is formed around the vent hole between the first section and the second section and has an inner perimeter directly connecting a bottom end of the first section and an outer perimeter directly connecting a top end of the second section. The second step is formed around the vent hole between the second section and the third section and having an inner perimeter directly connecting a bottom end of the second section and outer perimeter of the third section.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is schematic perspective view illustrating a first embodiment of a cell culture assisting apparatus according to the present disclosure.

FIG. 2 is cross-sectional view taken along line II-II in FIG. 1 showing the first embodiment.

FIG. 3 is schematic perspective view illustrating a second embodiment of a cell culture assisting apparatus according to the present disclosure.

FIG. 4 is a cross-sectional view taken along line IV-IV in FIG. 3 showing the second embodiment.

FIG. 5A is a perspective view illustrating a third embodiment of a cell culture assisting apparatus for assisting cell culture according to the present disclosure.

FIG. 5B is a bottom view of the third embodiment.

FIG. 6A illustrates a fourth embodiment of the present disclosure.

FIG. 6B is a bottom view of the fourth embodiment of the present disclosure.

FIG. 7 is an exploded perspective view illustrating a fifth embodiment of the present disclosure.

FIG. 8 is a cross-sectional view taken along line VI-VI in FIG. 7.

FIG. 9 is a cross-sectional view taken along line VI-VI in FIG. 7.

FIG. 10 is a cross-sectional view taken along line VI-VI in FIG. 7.

FIG. 11 illustrates fluid flow patterns which may be formed by the cell culture assisting apparatus of the fifth embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features May be oriented differently (e.g., rotated 90 degrees or at other orientations) and the spatially relative terms used herein may be interpreted accordingly.

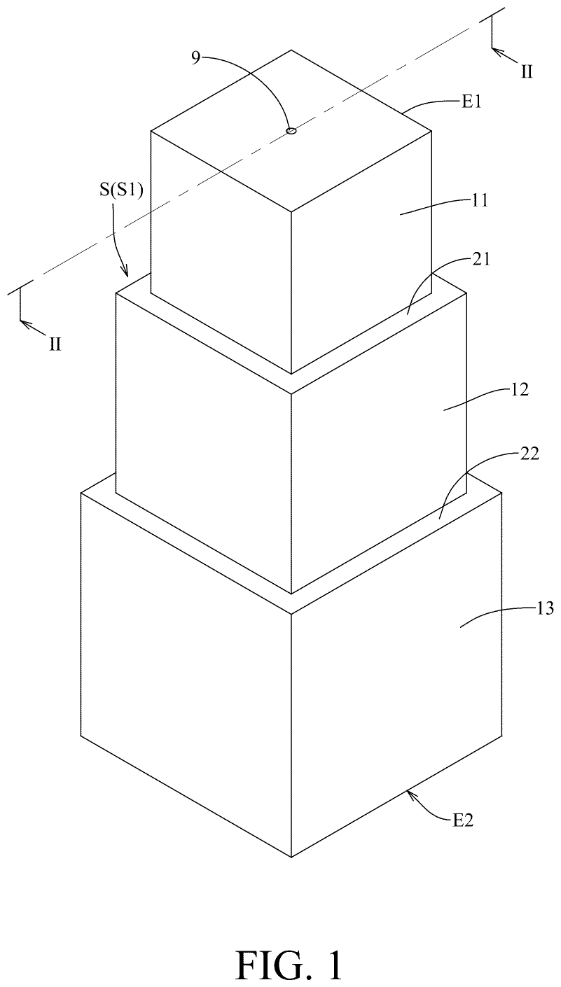

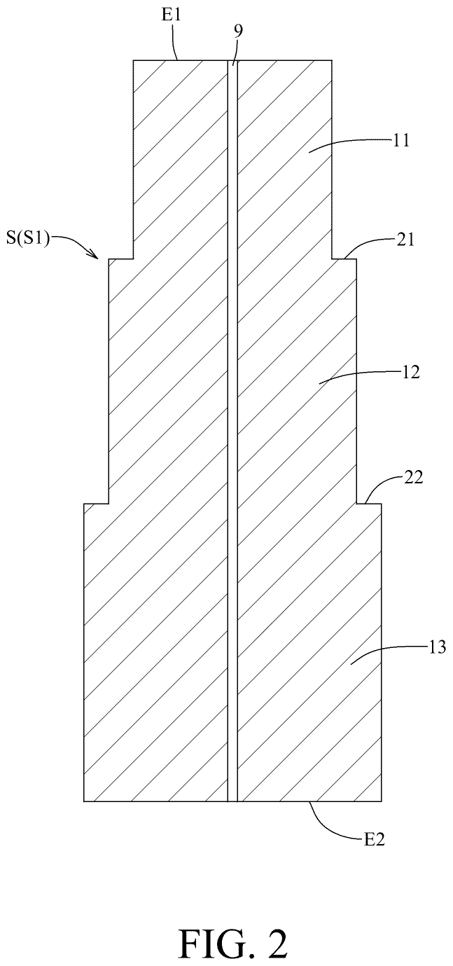

Referring to FIGS. 1 and 2, a first embodiment of a cell culture assisting apparatus includes a stepped structure(S), and a vent hole (9). The stepped structure(S) includes a first section 11, a second section 12, a third section 13, a first step 21, and a second step 22. The first section 12 surrounds the vent hole 9. The second section 12 surrounds the vent hole 9 below the first section 11 and is wider than the first section 11. The third section 13 surrounds the vent hole 9 below the second section 12 and is wider than the second section 12.

In this embodiment, the first section 11, the second section 12, and the third section 13 are each in a shape of a rectangular column.

In the first embodiment, the first section 11 (i.e., both an inner surface and an outer surface thereof), the second section 12 (i.e., both an inner surface and an outer surface thereof), and the third section 13 (i.e., both an inner surface and an outer surface thereof) are made of metal. It is noted that in the first embodiment, the metal is copper. However, it should be noted that this is not a limitation of the disclosure, and in some embodiments, only the first section 11 may be made of metal.

The first step 21 is formed around the vent hole 9 between the first section 11 and the second section 12. The second step 22 is formed around the vent hole 9 between the second section 12 and the third section 13. In this embodiment, the first step 21, and the second step 22 both have a rectangular ring shape, and an inner diameter of the second step 22 is equal to an outer diameter of the first step 21.

In the first embodiment, an inner perimeter of the first step 21 is connected to the outer surface of the first section 11, and an outer perimeter of the first step 21 is connected to the outer surface of the second section 12. The inner perimeter of the second step 22 is connected to the outer surface of the second section 12, and an outer perimeter of the second step 22 is connected to the outer surface of the third step 13. In other words, the inner perimeter of the first step 21 directly connects a bottom end of the first section 11; the outer perimeter of the first step 21 directly connects a top end of the second section 12. The inner perimeter of the second step 22 directly connects a bottom end of the second section 12, and the outer perimeter of the second step 22 directly connects a top end of the third section 13. The stepped structure(S) has an outer terrace surface (S1) that faces outwardly of the vent hole 9 and that is composed of outer surfaces of the first, second and third sections 11, 12, 13 and the first and second steps 21, 22. In other words, the outer surface of the first section 11, the first step 21, the outer surface of the second section 12, the second step 22, and the outer surface of the third step 13 are arranged relative to each other as such so as to achieve the overall effect of the stepped structure(S).

The stepped structure(S) has a top end (E1), and a bottom end (E2). The vent hole 9 extends through the stepped structure(S) and opens at the bottom and top ends (E1, E2) of the stepped structure(S). In the first embodiment, the vent hole 9 is in the shape of cylinder.

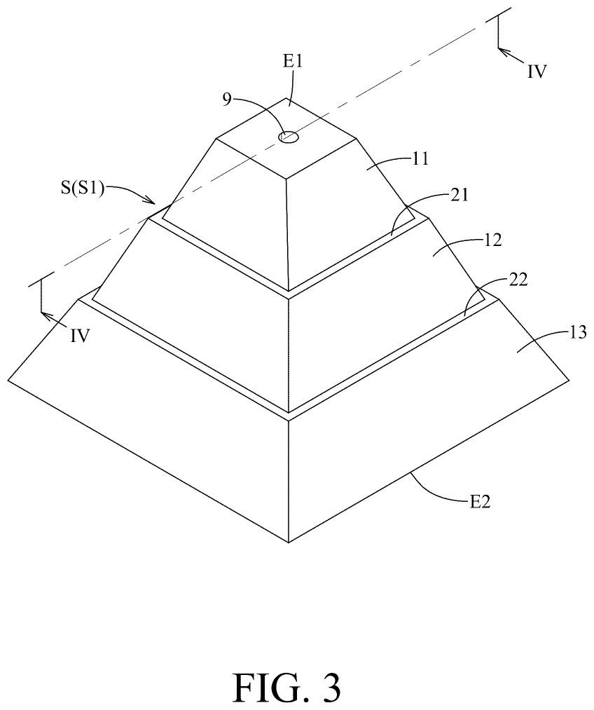

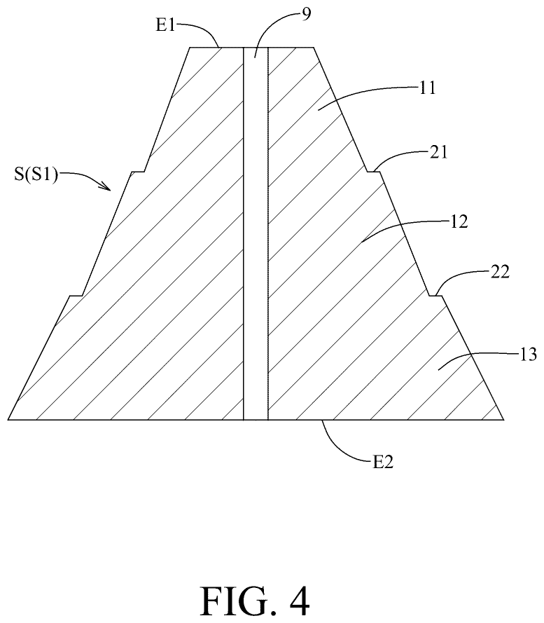

Referring to FIGS. 3 and 4, a second embodiment of the cell culture assisting apparatus capable of assisting cell culture is similar to the first embodiment; however, the differences therebetween are that in the second embodiment, the first section 11, the second section 12, and the third section 13 are each in the shape of a truncated pyramid. Additionally, in the second embodiment, the inner perimeter of the second step 22 is greater than the outer perimeter of the first step 21.

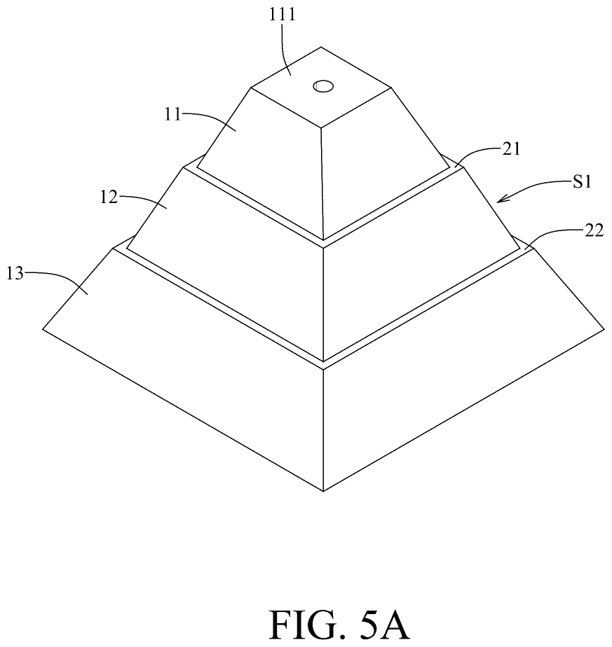

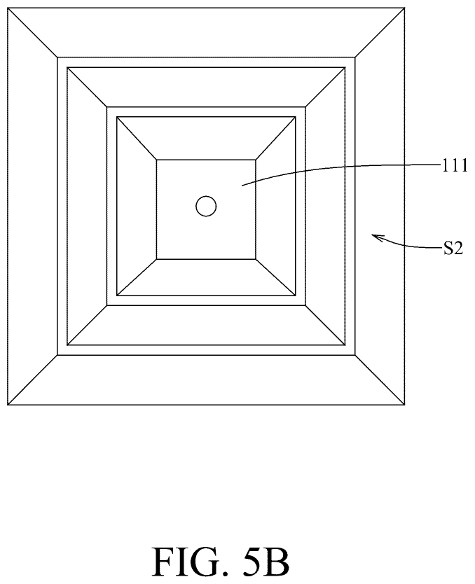

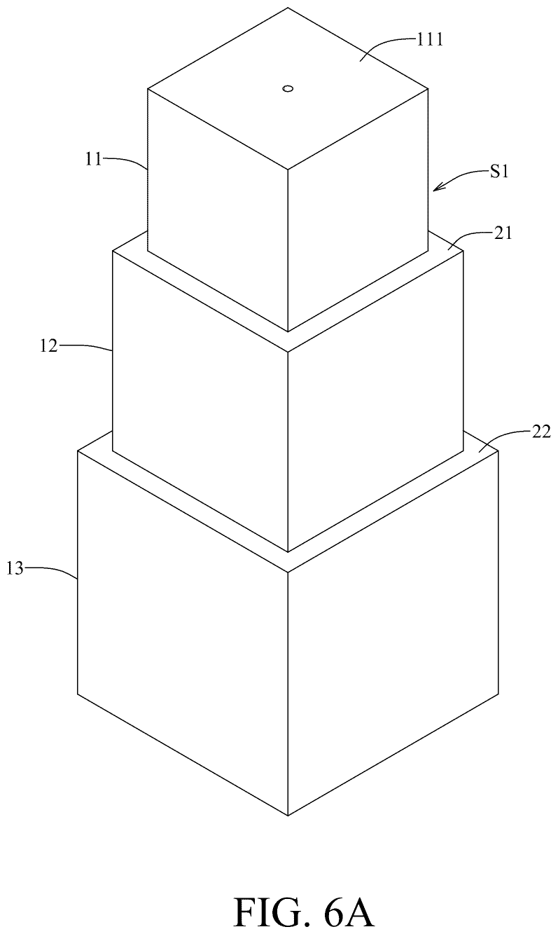

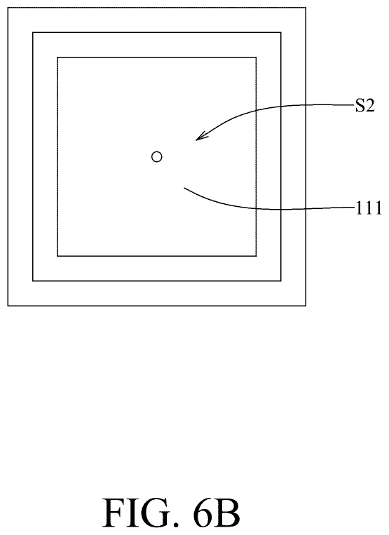

FIGS. 5A and 5B show a third embodiment of the present disclosure. FIGS. 6A and 6B show a fourth embodiment. The third and the fourth embodiments are basically similar to the first embodiment and the second embodiment, respectively. However, in each of the third and fourth embodiments, the stepped structure(S) has both an outer terraced surface (S1) and an inner terraced surface (S2). The outer terraced surface (S1) is composed of outer surfaces of the first, second and third sections 11, 12, 13, and the first and second steps 21, 22. The inner terraced surface (S2) is composed of inner surfaces of the first, second and third sections 11, 12, 13 and the first and second steps 21, 22.

Moreover, in the inner terraced surface (S2), the inner perimeter of the first step 21 is connected to the inner surface of the first section 11, and the outer perimeter of the first step 21 is connected to the inner surface of the second section 12. The inner perimeter of the second step 22 is connected to the inner surface of the second section 12, the outer perimeter of the second step 22 is connected to the inner surface of the third section 13. In this way, the inner surface of the first section 11, the first step 21, the inner surface of the second section 12, the second step 22, and the inner surface of the third section 13 collectively define the vent hole 9 and cooperatively form the stepped structure(S).

In addition, in each of the third and fourth embodiments, the first section 11 has a top cap portion 111. The top cap portion 111 is formed on one side of the first section 11 away from the first step 21 and has an inner surface connected to the inner terraced surface (S2) and an outer surface connected to the outer terraced surface (S1).

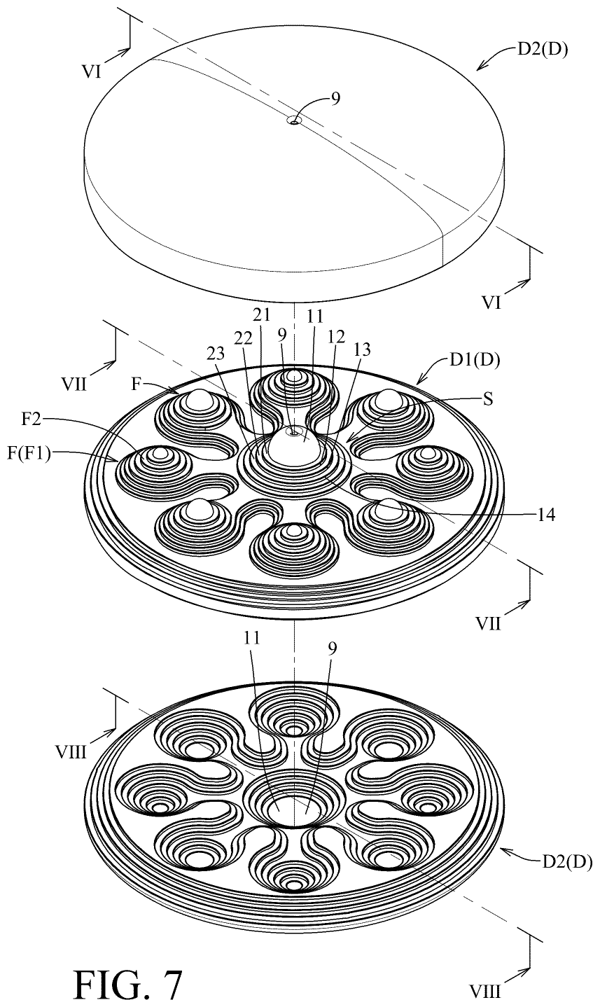

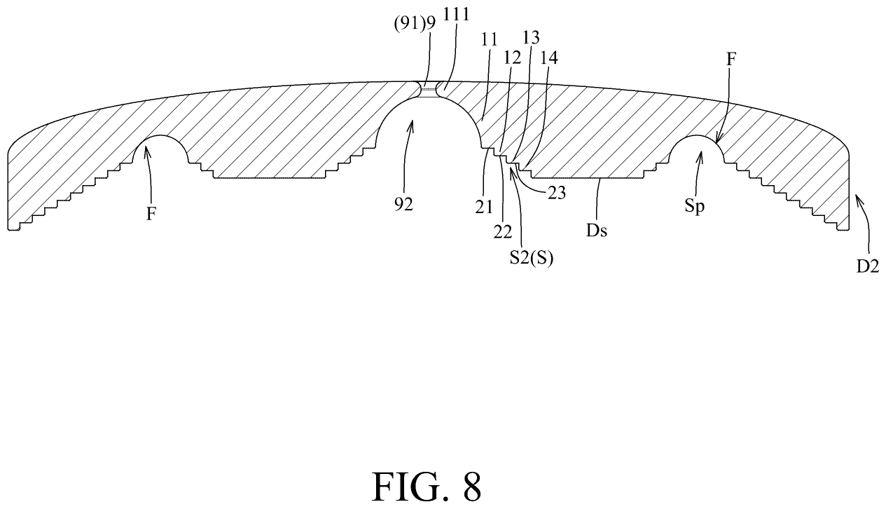

FIGS. 7 to 10 show a fifth embodiment of the cell culture assisting apparatus capable of assisting cell culture according to the present disclosure. The cell culture assisting apparatus of this embodiment includes three dishes (D) which are stacked together. One of the dishes (D) (hereinafter referred to as D1) is disposed between the remaining two of the dishes (D) (hereinafter referred to as D2). Referring to FIGS. 8, 9 and 10, each of the dishes (D1) and (D2) has a middle region, a lateral region surrounding the middle region, a stepped structure (S) formed in the middle region, and a plurality of stepped formations (F) that are disposed in the lateral region around the stepped structure(S). The stepped formations (F) are formed into a multi-petal-shaped pattern which surrounds said stepped structure(S) (see FIG. 7).

Each of the stepped formations (F) has an interior space, a terraced surface (F1) that includes a plurality of terrace steps (F2) surrounding the interior space. The terrace steps (F2) of the terraced surfaces (F1) of the stepped formations (F) are connected to each other in series in such a manner that the terrace steps (F2) of the stepped formations (F) extend continuously to form loops that surround the stepped structure(S), each of the loops having a multi-petal-shaped pattern.

The dish (D1) is slightly different from the dishes (D2). In particular, the dish (D1) has the stepped structure(S) and the stepped formations (F) protruding from a surface (Ds) of the dish (D1), whereas each of the dishes (D2) has the stepped structure(S) and the stepped formations (F) indented from the surface (Ds) of the dish (D2).

Compared to the previous embodiments shown in FIGS. 1 to 6, the stepped structure(S) in the fifth embodiment additionally includes a fourth section 14 (see FIGS. 8, 9, 10) that surrounds the vent hole 9 below the third section 13, and that is connected to a bottom end of the third section 13 remote from the second section 12, and a third step 23 that is formed between the third section 13 and the fourth section 14, and that has an inner diameter that is no less than an outer diameter of the second step 22.

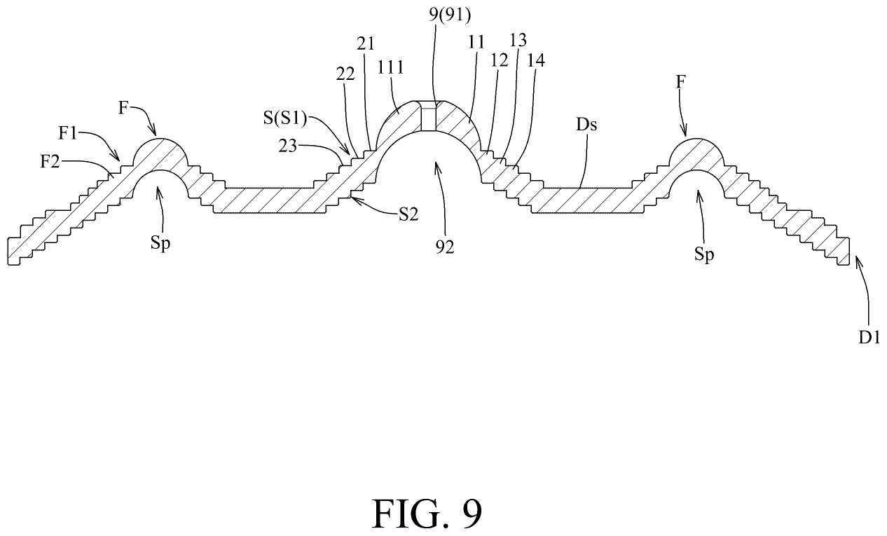

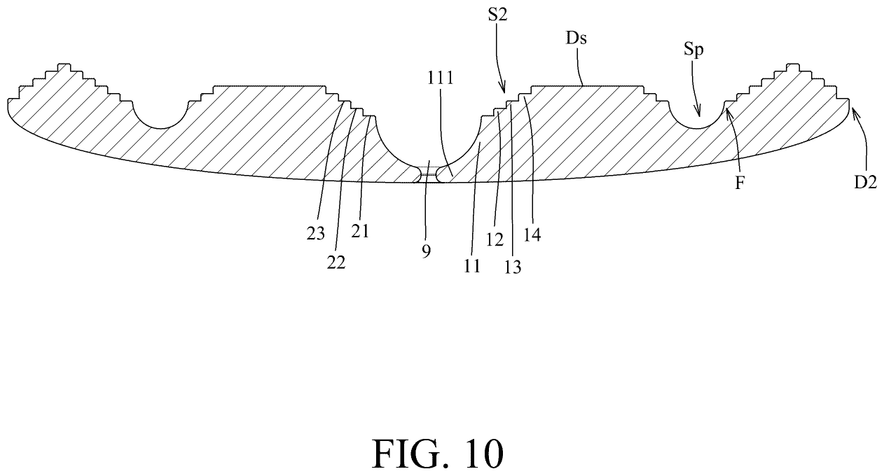

Referring again to FIGS. 7 to 10, the first section 11 of the stepped structure(S) of each of the dishes (D1, D2) has a top cap portion 111 that is formed on one side of the first section 11 away from the first step 21, As shown in FIG. 9, in the dish (D1), the top cap portion 111 has a convex outer surface connected to the outer terraced surface (S1). The vent hole 9 has a narrow hole section 91 extending upwardly through the top cap portion 111, and an enlarged hole section 92 extending downwardly from the narrow hole section 91. The top cap portion 111 further has a concave inner surface that is connected to the inner terraced surface (S2) of the stepped structure(S). Compared to the dish (D1) shown in FIG. 9, the top cap portion 111 in each dish (D2) of FIG. 8 or 10 only has the inner convex outer surface (S2) connected to the inner terraced surface (S2).

In the fifth embodiment, the first section 11 (i.e., both the inner surface and the outer surface), the second section 12 (i.e., both the inner surface and the outer surface), the third section 13 (i.e., both the inner surface and the outer surface), and the fourth section 14 (i.e., both an inner surface and an outer surface) are made of metal. It should be noted that in the third embodiment, the fourth embodiment, and the fifth embodiment, the metal is copper.

In the fifth embodiment, the first step 21 is a circular annular ring, the second step 22 is a circular annular ring, and the third step 23 is a circular annular ring. The third step 23 has an inner perimeter that is equal to the outer perimeter of the second step 22.

In addition, in each of dishes (D1, D2) of the fifth embodiment, the first section 11 has a top cap portion 111 that is formed on one side of the first section 11 away from the first step 21 and has an inner surface connected to the inner terraced surface (S2) and an outer surface connected to the outer terraced surface (S1). Moreover, the top cap portion 111 has a concave inner surface connected to the terraced surface (S2).

In the fifth embodiment, each of the stepped formations (F) has a configuration that is generally similar to that of the stepped structure(S). However, each stepped formation (F) has a top cap portion 111 which does not have the narrow hole portion 92 of the vent hole 9 of the top cap portion 111 of the stepped structure(S). In addition, each stepped formation (F) is connected to adjacent stepped formations (F).

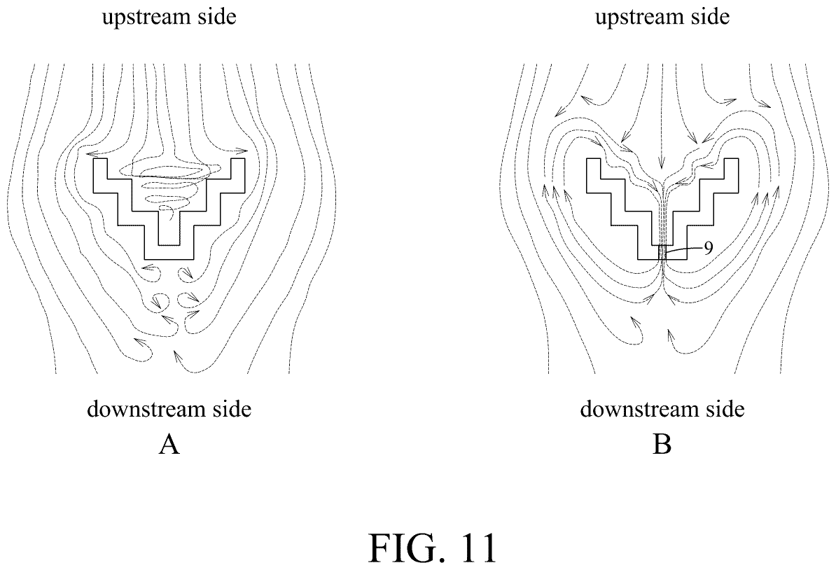

FIG. 11 illustrates a fluid flow pattern (A) which may be generated in each stepped formation (F) and a fluid flow pattern (B) which may be generated in the stepped structure(S) of the fifth embodiment. It was observed that Karman vortices formed in the fluid flow patterns (A) and (B) have an increased stability and have improved energy conversion efficiency of the fluid flow. It should be noted that the dimensions of the stepped structure(S) and the vent hole (9) of the first embodiment may be adjusted to facilitate the generation of the Karman vortex in the fluid flow. More specifically, when the fluid flow passes through the stepped structure(S) vortices will be formed at the edges of the stepped structure(S). These vortices alternately form and shed from the stepped structure(S), thereby forming the Karman vortex which is conducive to efficient energy transfer.

### Cell Culture Experiment

A set of six cell culture assisting apparatuses of the fifth embodiment are arranged in a 2×3 matrix. A pair of petri dishes are disposed respectively directly above and below the 2×3 matrix set. The petri dishes are inoculated with adult stem cells (ASCs) extracted from fat cells and used as experimental groups (Ex1, Ex2). Another petri dish is inoculated with ASCs as a control group. It should be noted that Ex1, Ex2, and the control group are inoculated with the same amount of ASCs.

A culture fluid containing 10% Fetal Bovine Serum (FBS) is added to each of Ex1, Ex2, and the control group. Ex1, Ex2, and the control group are placed in a cell culture incubator set to 37° C., 5% CO₂, and 20% relative humidity and incubated for 24 hours. Afterwards, MTT assay is used to analyze the cell growth rate of ASCs (n=3) in Ex1, Ex2, and the control group at 0 h, 0.5 h, 2 h, 6 h, 16 h, 24 h. The results are displayed in table 1 below.

TABLE 1

| | 0 h | 0.5 h | 2 h | 6 h |
|---|---|---|---|---|
| control | 1.000 ± 0.043 | 1.000 ± 0.014 | 1.000 ± 0.020 | 1.000 ± 0.007 |
| group | | | | |
| Ex1 above | 0.964 ± 0.079 | 1.022 ± 0.004 | 1.082 ± 0.009 | 1.079 ± 0.024 |
| | (p = 0.712) | (p = 0.205) | (p = 0.020) | (p = 0.035) |
| Ex2 below | 0.966 ± 0.033 | 0.996 ± 0.021 | 1.110 ± 0.010 | 1.084 ± 0.006 |
| | (p = 0.570) | (p = 0.882) | (p = 0.008) | (p = 0.001) |

| | 16 h | 20 h | 24 h |
|---|---|---|---|
| control | 1.000 ± 0.005 | 1.000 ± 0.014 | 1.000 ± 0.017 |
| group | | | |
| Ex1 above | 1.112 ± 0.019 | 1.108 ± 0.009 | 1.239 ± 0.014 |
| | (p = 0.005) | (p = 0.003) | (p = 0.000) |
| Ex2 below | 1.115 ± 0.021 | 1.123 ± 0.022 | 1.244 ± 0.034 |
| | (p = 0.006) | (p = 0.010) | (p = 0.003) |

From Table 1 it may be inferred that from 2 h to 24 h of incubation, Ex1 and Ex2 show significantly higher cell growth rate of ASCs compared to the control group (i.e., from 1.079 times to 1.244 times the growth rate of the control group, p≤0.020).

In summary of the above, in the cell culture assisting apparatus according to the present disclosure, by virtue of including the first section 11, the second section 12, the third section 13, the first step 21, the second step 22, and the vent hole 9, fluid flow in the vicinity of the cell culture assisting apparatus may increase cell growth rate, thereby achieving the object of the disclosure.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A cell culture assisting apparatus comprising:
a stepped structure having a top end, and a bottom end; and
a vent hole extending though said stepped structure and opening at said bottom and top ends;
said stepped structure further having:
a first section surrounding said vent hole and defining said top end;
a second section surrounding said vent hole below said first section and being wider than said first section;
a third section that surrounds said vent hole below said second section and being wider than said second section;
a first step formed around said vent hole between said first section and said second section and having an inner perimeter directly connecting a bottom end of said first section and an outer perimeter directly connecting a top end of said second section; and
a second step formed around said vent hole between said second section and said third section and having an inner perimeter directly connecting a bottom end of said second section and an outer perimeter of said third section.

2. The cell culture assisting apparatus as claimed in claim 1, wherein:
said stepped structure has an outer terraced surface that faces outwardly of said vent hole and that is composed of outer surfaces of said first, second and third sections and said first and second steps;
said inner perimeter of said first step is connected to said outer surface of said first section;
said inner perimeter of said second step is connected to said outer surface of said second section; and
said outer perimeter of said second step is connected to said outer surface of said third section.

3. The cell culture assisting apparatus as claimed in claim 2, wherein:
said first section has a top cap portion that is formed on one side of said first section away from said first step and that has an outer surface connected to said outer terraced surface; and
said vent hole has a narrow hole section extending upwardly through said top cap portion, and an enlarged hole section extending downwardly from said narrow hole section.

4. The cell culture assisting apparatus as claimed in claim 1, wherein:
said stepped structure has an inner terraced surface that faces toward said vent hole and is composed of inner surfaces of said first, second and third sections and said first and second steps;
said first step has said inner perimeter that is connected to said inner surface of said first section; and
said second step has said inner perimeter that is connected to said inner surface of said second section, and said outer perimeter that is connected to said inner surface of said third section.

5. The cell culture assisting apparatus as claimed in claim 4, wherein:
said first section has a top cap portion that is formed on one side of said first section away from said first step and that has an inner surface connected to said outer terraced surface; and
said vent hole has a narrow hole section extending upwardly through said top cap portion, and an enlarged hole section extending downwardly from said narrow hole section.

6. The cell culture assisting apparatus as claimed in claim 1, wherein an inner diameter of said second step is equal to an outer diameter of said first step.

7. The cell culture assisting apparatus as claimed in claim 3, wherein said top cap portion has a convex outer surface connected to said outer terraced surface.

8. The cell culture assisting apparatus as claimed in claim 5, wherein said top cap portion has a concave inner surface connected to said inner terraced surface.

9. The cell culture assisting apparatus as claimed in claim 1, wherein an inner surface of said first section is made of a metal.

10. The cell culture assisting apparatus as claimed in claim 1, wherein said first section, said second section, and said third section are made of metal.

11. The cell culture assisting apparatus as claimed in claim 1, further comprising:
a fourth section surrounding said vent hole below said third section; and
a third step formed between said third section and said fourth section, and having an inner diameter that is no less than an outer diameter of said second step.

12. The cell culture assisting apparatus as claimed in claim 1, wherein said stepped structure has an outer terraced surface and an inner terraced surface, said outer terraced surface being composed of outer surfaces of said first, second and third sections and said first and second step, said inner terraced surface being composed of inner surfaces of said first, second and third sections and said first and second steps.

13. The cell culture assisting apparatus as claimed in claim 1, further comprising at least one dish that includes a middle region and a lateral region surrounding said middle region, said middle region being formed with said stepped structure, said lateral region having a plurality of stepped formations that are disposed around said stepped structure, each of said stepped formations having a terraced surface that includes a plurality of terrace steps.

14. The cell culture assisting apparatus as claimed in claim 13, wherein said stepped formations are formed in a multi-petal-shaped pattern that surrounds said stepped structure.

15. The cell culture assisting apparatus as claimed in claim 14, wherein said terrace steps of said terraced surfaces of said stepped formations are connected to each other in such a manner that said terrace steps of said stepped formations extend continuously to form loops that surround said stepped structure, each of said loops having said multi-petal-shaped pattern.

16. The cell culture assisting apparatus as claimed in claim 14, wherein said stepped structure and said stepped formations indent from a surface of said dish.

17. The cell culture assisting apparatus as claimed in claim 14, wherein said stepped structure and said stepped formations protrude from a surface of said dish.

18. The cell culture assisting apparatus as claimed in claim 14, wherein said at least one dish includes three dishes which are stacked together, one of said dishes being disposed between the remaining two of said dishes, said one of said dishes having said stepped structure and said stepped formations which indent from said surface of said dish, each of said remaining two of said dishes having said stepped structure and said stepped formations which protrude from said surface of said dish.
